(19) United States
(12) Patent Application Publication
(10) Pub. No.: US 2003/0212312 A1
(43) Pub. Date: Nov. 13, 2003

(54) Low noise patient cable

(76) Inventors: James P. Coffin IV, Mission Viejo, CA (US); John Schmidt, Lake Forest, CA (US); Yassir Abdul-Hafiz, Irvine, CA (US); Jose Quinones, Norwalk, CA (US); Deshan L. Atapattu, Chatsworth, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/325,602

(22) Filed: Dec. 19, 2002

Related U.S. Application Data
(60) Provisional application No. 60/346,725, filed on Jan. 7, 2002.

(51) Int. Cl. A61B 5/00

(52) U.S. Cl. 600/300

(57) ABSTRACT

A low noise patient cable has a plurality of emitter wires configured to communicate a drive signal between a monitor and at least one emitter. A plurality of detector wires is also configured to communicate a physiological signal between a detector responsive to the emitter and the monitor. A polymer layer is disposed around, and adapted to conduct a triboelectric charge away from, the detector wires.

### CROSS-REFERENCE TO RELATED APPLICATIONS

The present application claims priority benefit under 35 U.S.C. §119(e) from U.S. Provisional Application No. 60/346,725, filed Jan. 7, 2002, entitled “Low Noise Patient Cable,” which is incorporated herein by reference.

### BACKGROUND OF THE INVENTION

Pulse oximetry is a widely accepted noninvasive procedure for measuring the oxygen saturation level of arterial blood, an indicator of a person's oxygen supply. Early detection of low blood oxygen level is of crucial importance in the medical field, for example in critical care and surgical applications, because an insufficient supply of oxygen can result in brain damage and death in a matter of minutes. A pulse oximetry system consists of a sensor applied to a patient, a monitor, and a patient cable connecting the sensor and the monitor. The monitor may be a standalone device or may be incorporated as a module or built-in portion of a multiparameter patient monitoring system. A monitor typically provides a numerical readout of the patient's oxygen saturation, a numerical readout of pulse rate, and an audible indication of each pulse. In addition, the monitor may display the patient's plethysmograph, which provides a visual display of the patient's pulse contour and pulse rate.

### SUMMARY OF THE INVENTION

One aspect of a low noise patient cable is a plurality of emitter wires configured to communicate a drive signal between a monitor and at least one emitter. A plurality of detector wires is also configured to communicate a physiological signal between a detector, which is responsive to energy received from the at least one emitter, and the monitor. A polymer layer is disposed around, and adapted to conduct a triboelectric charge away from, the detector wires. In one embodiment, the detector wires are configured as a twisted pair and the polymer layer is coextruded with the twisted pair. In a particular embodiment, the polymer layer is a conductive PVC, which may be coextruded to a diameter in the range of about 0.055 to about 0.061 inches and that may also utilize a flexible conductive vinyl.

Another aspect of a low noise patient cable is a method comprising the steps of twisting a pair of detector wires, coextruding the wires with a conductive polymer to form a polymer layer disposed around the insulator of each of the wires, extending a pair of emitter wires proximate the detector wires and disposing an outer jacket around the detector wires and the emitter wires so as to form a patient cable. In one embodiment, the method further comprises the steps of disposing an inner shield around the polymer layer and disposing an inner jacket around the inner shield, where the inner shield and the inner jacket are configured so as to be between the detector wires and the emitter wires. In a particular embodiment, the method further comprises the step of disposing an outer shield around the inner jacket and the emitter wires, where the outer shield is configured so as to be encased by the jacket.

Yet another aspect of a low noise patient cable is a detector wire means for conducting a physiological signal between a sensor and a monitor. A polymer means for conducting triboelectric charge is coextruded with the detector wire means. Further an emitter wire means for conducting a drive signal between the monitor and the sensor is jacketed with the detector wire means. In one embodiment, the low noise patient cable further comprises a first conductive means for shielding the detector wire means, which is jacketed with the detector wire means, and a second conductive means for shielding the emitter wire means, which is jacketed with the emitter wire means, the detector wire means and the first conductive means.

### BRIEF DESCRIPTION OF THE DRAWINGS

FIG. 1 is a schematic diagram of a prior art pulse oximetry system;

FIGS. 2A-B are a cross-section and cutaway side-view, respectively, of a prior art patient cable; and

FIGS. 3A-B are a cross-section and cutaway side-view, respectively, of a low noise patient cable.

### DETAILED DESCRIPTION OF THE PREFERRED EMBODIMENTS

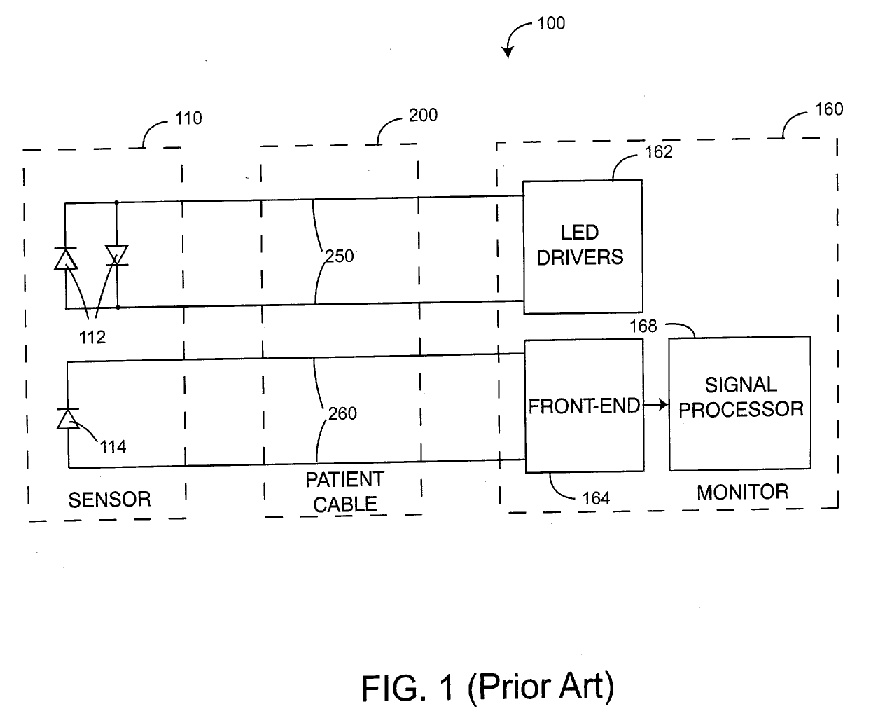

FIG. 1 illustrates the functions of a pulse oximetry system 100. The sensor 110 has both red and infrared (IR) light-emitting diode (LED) emitters 112 and a photodiode detector 114. The monitor 160 has LED drivers 162, a front-end 164 and a signal processor 168. The monitor 160 determines oxygen saturation by computing the differential absorption by arterial blood of the two wavelengths emitted by the sensor emitters 112, as is well-known in the art. The LED drivers 162 provide drive current which alternately activates the red and IR LED emitters 112. The patient cable 200 conducts the LED drive current over drive wires 250 connecting the LED drivers 162 to the LED emitters 112. The photodiode detector 114 generates a signal corresponding to the red and IR light energy attenuated from transmission through a tissue site. The patient cable 200 conducts the detector signal over detector wires 260 connecting the detector 114 to the front-end 164. The front-end 164 has input circuitry for amplification, filtering and digitization of the detector signal, which is then input to the signal processor 168. The signal processor 168 calculates a ratio of detected red and infrared intensities, and an arterial oxygen saturation value is empirically determined based on that ratio. A pulse oximetry sensor is described in U.S. Pat. No. 6,088,607 entitled Low Noise Optical Probe, which is assigned to the assignee of the present invention and incorporated by reference herein. A pulse oximetry signal processor is described in U.S. Pat. No. 6,081,735 entitled Signal Processing Apparatus, which is assigned to the assignee of the present invention and incorporated by reference herein.

In a pulse oximetry system, the detector typically generates a low-level signal that is susceptible to corruption from various noise sources, such as electromagnetic interference (EMI) and internal noise sources that originate in the sensor, the patient cable and the monitor. One internal noise source is due to the triboelectric effect, which is the static charge generated when two materials are rubbed together. Triboelectric noise is induced in the detector signal when, for example, the detector wires of the patient cable rub together, such as when the patient cable is flexed or is impacted. Triboelectric noise spikes can be orders of magnitude larger than the detector signal.

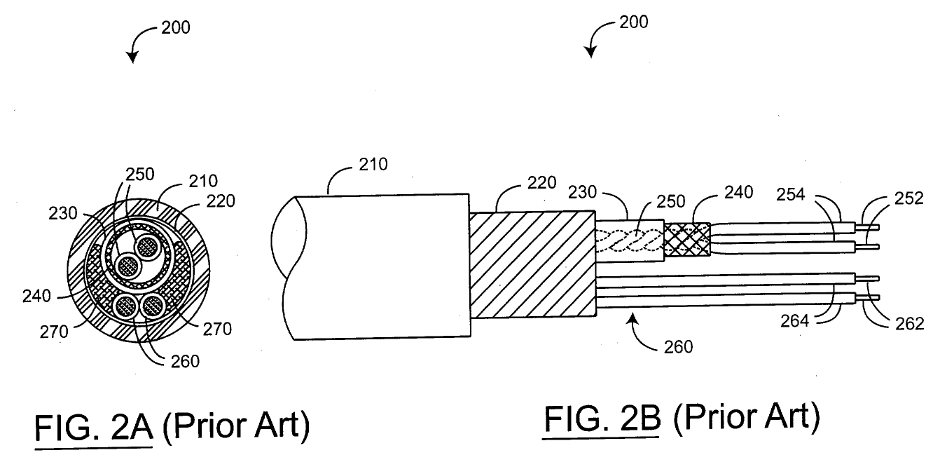

FIGS. 2A-B illustrate a patient cable 200 designed for a pulse oximetry system 100 (FIG. 1). The patient cable 200 has an outer jacket 210, an outer shield 220, an inner jacket 230, a graphite coating 240, detector wires 250 configured as a twisted pair, emitter wires 260 and textile fillers 270. The twisted pair 250 has detector conductors 252 and associated insulation 254. The emitter wires 260 have emitter conductors 262 and associated insulation 264. The shield 220 and the twisted pair configuration of the detector wires 250 reduce noise due to EMI and crosstalk. Because of the proximity of the twisted pair insulation 254, however, the detector wires 250 are prone to rubbing and, hence, triboelectric noise. The graphite coating 240 provides a conductive layer along the outside of the detector wires 240, reducing triboelectric noise by draining the triboelectric induced charge away from the detector wire insulation 254.

The coating 240 is formed by drawing the twisted pair 250 through a solvent bath containing graphite. The solvent is allowed to evaporate, depositing the conductive graphite coating 240 on the twisted pair 250. A deposited graphite coating 240, however, has several drawbacks. The coating 240 is difficult to precisely manufacture because the deposition process is difficult to control. As a result, the cable 200 itself is relatively expensive to manufacture. Also, preparation of the cable 200 for connector attachment involves cutting and stripping the cable layers to expose the conductors 252, 262, which are difficult procedures to perform. In particular, the deposited coating 240 has to be selectively cleaned-off with a solvent and mechanical abrasion to expose the conductor ends 252, which is time consuming and which may subject the cable 200 to damage.

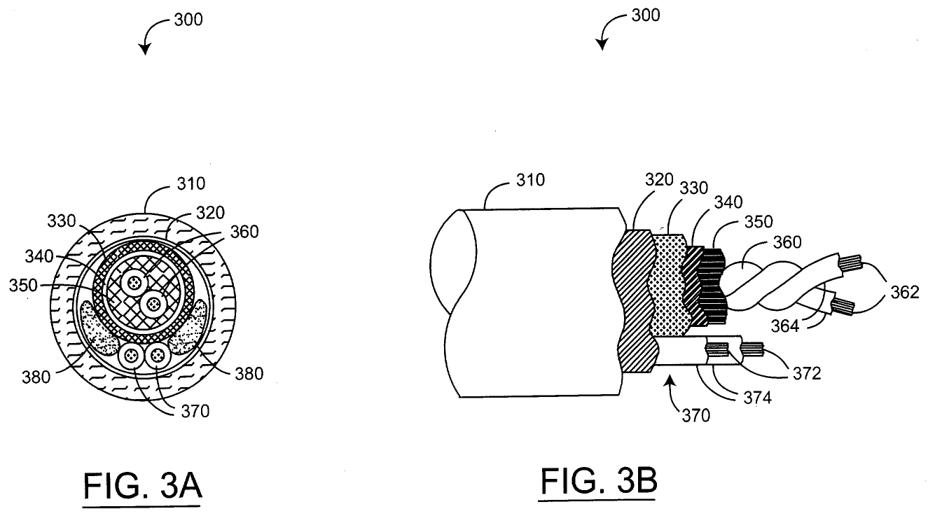

FIGS. 3A-B illustrates a low noise patient cable 300, which has an outer jacket 310, an outer shield 320, an inner jacket 330, an inner shield 340, a polymer layer 350, detector wires 360 configured as a twisted pair, emitter wires 370 and textile fillers 380. The twisted pair 360 has detector conductors 362 and associated insulation 364. The emitter wires 370 have emitter conductors 372 and associated insulation 374. The low noise patient cable 300 functions in a pulse oximetry system 100 (FIG. 1) in a manner similar to that of the patient cable 200 (FIG. 2) described above. In particular, the emitter wires 370 electrically connect the LED drivers 162 (FIG. 1) to the LEDs 112 (FIG. 1), and the twisted pair 360 electrically connects the detector 114 (FIG. 1) to the monitor front-end 164 (FIG. 1). Further, the shields 320, 340 and twisted pair 360 reduce EMI and crosstalk. The polymer layer 350, however, is advantageously disposed around the detector wires 360 instead of a graphite coating as described with respect to FIG. 2, above. The polymer layer 350 is formed by coextruding the twisted pair 360 with a conductive polymer. In one embodiment, the polymer layer 350 is a conductive PVC. In a particular embodiment, the conductive PVC utilizes a flexible conductive vinyl compound, such as Abbey #100-1 available from Abbey Plastic Corporation and is coextruded to a diameter in the range of about 0.058±0.003 inches.

A coextruded conductive polymer has several advantages over a deposited graphite coating for reducing triboelectric noise. As with the graphite coating, the polymer layer 350 drains the triboelectric induced charge away from the detector wire insulation 364. The coextrusion process, however, is easier to control and less expensive accordingly. Further, during cable preparation for connector attachment the polymer layer 350 can be easily cut from the twisted pair 360. In addition, better triboelectric noise reduction can be achieved with the polymer layer 350 than with a graphite coating.

In addition to the foregoing, disposing the polymer layer 350 around the twisted pair of detector wires 360 has several advantages over disposing the polymer layer 350 around individual wires. For example, disposal around the twisted pair can be less expensive than disposal around individual wires and can produce an end product cable having a smaller diameter. Moreover, disposal around the twisted pair in the embodiment of the low noise patient cable 300 being used for at least pulse oximetry, can increase the eventual signal quality output from signal processing circuitry, such as, for example, a differential amplifier. For example, formation of the polymer layer 350 in a manner that maintains the close physical proximity of the twisted pair tends to ensure external noise applied to the patient cable 300 is applied substantially equally (or common) to each conductor of the twisted pair. Thus, the differential amplifier (not shown) of the monitor 160 can effectively filter the applied external noise through, for example, the amplifier's common mode rejection.

The low noise patient cable has been disclosed in detail in connection with various embodiments. These embodiments are disclosed by way of examples only and are not intended to limit the scope of the claims that follow. One of ordinary skill in the art will appreciate many variations and modifications.

What is claimed is:

1. A low noise patient cable comprising:
a plurality of emitter wires configured to communicate a drive signal between a monitor and at least one emitter;
a plurality of detector wires configured to communicate a physiological signal between a detector, which is responsive to energy received from said at least one emitter, and said monitor; and
a polymer layer disposed around at least a pair of said detector wires, said polymer layer adapted to conduct a triboelectric charge away from said detector wires.

2. The low noise patient cable according to claim 1 wherein said pair of detector wires is configured as a twisted pair and said polymer layer is coextruded with said twisted pair.

3. The low noise patient cable according to claim 2 wherein said polymer layer is a conductive PVC.

4. The low noise patient cable according to claim 3 wherein said conductive PVC is coextruded to a diameter in the range of about 0.055 to about 0.061 inches.

5. The low noise patient cable according to claim 4 wherein said conductive PVC is a flexible conductive vinyl compound.

6. A cabling method comprising the steps of:
twisting a pair of detector wires;
coextruding said wires with a conductive polymer to form a polymer layer disposed around said insulator of each of said wires;
extending a pair of emitter wires proximate said detector wires; and
disposing an outer jacket around said detector wires and said emitter wires so as to form a patient cable.

7. The cabling method according to claim 6 further comprising the steps of:
disposing an inner shield around said polymer layer; and
disposing an inner jacket around said inner shield,
said inner shield and said inner jacket configured so as to be between said detector wires and said emitter wires.

8. The cabling method according to claim 7 further comprising the step of disposing an outer shield around said inner jacket and said emitter wires, said outer shield configured so as to be encased by said jacket.

9. A patient cable comprising:
a detector wire means for conducting a physiological signal between a sensor and a monitor;
a polymer means for conducting triboelectric charge coextruded with said detector wire means; and
an emitter wire means for conducting a drive signal between said monitor and said sensor jacketed with said detector wire means.

10. The patient cable according to claim 9 further comprising:
a first conductive means for shielding said detector wire means jacketed with said detector wire means; and
a second conductive means for shielding said emitter wire means jacketed with said emitter wire means, said detector wire means and said first conductive means.
